(19) United States
(12) Patent Application Publication
(10) Pub. No.: US 2008/0287486 A1
(43) Pub. Date: Nov. 20, 2008

(54) Prokinetic agent for bowel preparation

(75) Inventors: Michael Kitt, Mountain View, CA (US); Mchael Goldberg, San Francisco, CA (US)

Correspondence Address: THERAVANCE, INC., 901 GATEWAY BOULEVARD, SOUTH SAN FRANCISCO, CA, 94080, US

(73) Assignee: Theravance, Inc.

(21) Appl. No.: 12/152,528

(22) Filed: May 15, 2008

Related U.S. Application Data
(60) Provisional application No. 60/930,604, filed on May 17, 2007.

(51) Int. Cl. A61K 31/46; A61P 1/10

(52) U.S. Cl. 514/304

(57) ABSTRACT

The invention provides methods of bowel preparation before a diagnostic, surgical or therapeutic procedure, in particular, bowel preparation before a colonoscopy procedure, using a 5-HT₄ receptor agonist as a prokinetic agent.

### CROSS-REFERENCE TO RELATED APPLICATIONS

This application claims the benefit of U.S. Provisional Application No. 60/930,604, filed on May 17, 2007, the disclosure of which is incorporated herein by reference in its entirety.

### BACKGROUND OF THE INVENTION

1. Field of the Invention

The invention is directed to methods of bowel preparation before a diagnostic, surgical or therapeutic procedure, in particular, bowel preparation before a colonoscopy procedure, using a 5-HT₄ receptor agonist as a prokinetic agent.

2. State of the Art

Colonoscopy is a widely used and effective procedure for colorectal cancer screening and surveillance as well as for assessing other gastrointestinal symptoms. However, successful visualization of the colon, essential for the detection of suspicious lesions, depends on adequate bowel preparation. Complete clearing of the bowel of fecal residue is required in preparation for colonoscopy, for other procedures such as a barium enema, or for gastrointestinal or other surgical procedures.

A number of different regimens are available for bowel cleansing. Commonly used bowel cleansing preparations include osmotically balanced polyethylene glycol (PEG) electrolyte solutions, osmotic laxatives, such as sodium phosphate, magnesium sulfate, magnesium citrate and mannitol, and stimulant laxatives, such as bisacodyl, senna, and sodium picosulfate. Most of these regimens require the patient to be limited to a clear liquid diet for 24 hours or more prior to the procedure and to consume large volumes of liquid, at least a gallon.

A recent consensus document prepared by gastrointestinal surgeons suggested that no preparation method currently available ideally satisfies all requirements for efficacious cleansing and patient tolerability (Wexner et al. (2007) Dis Colon Rectum 49 792-809). It has been found that a significant number of suboptimal or incomplete colonoscopies can be attributed to inadequate bowel preparation (Belsey et al., (2007) Aliment Pharmacol Ther 25, 373-384; Barkun et al. (2006) Can J Gastroenterol 20 (11), 699-710). Poor patient compliance with the currently used preparative regimens is a common cause of inadequate preparation (Barkun et al.; Delegge et al. (2005) Aliment Pharmacol Ther 21, 1491-1495).

Thus, there is a need for a method of cleansing the bowel prior to diagnostic or surgical procedures that is more tolerable to patients than the presently available procedures in order to improve patient compliance. It would be desirable for the method to provide complete evacuation and to act more rapidly than the currently used approaches

### SUMMARY OF THE INVENTION

The present invention provides a method of cleansing the bowel of a patient in preparation for a diagnostic, surgical, or therapeutic procedure of the colon, the method comprising administering an effective amount of 1-isopropyl-2-oxo-1,2-dihydroquinoline-3-carboxylic acid {(1S,3R,5R)-8-[(R)-2-hydroxy-3-(methanesulfonyl-methyl-amino)propyl]-8-azabicyclo[3.2.1]oct-3-yl}amide, hereinafter compound 1, or a pharmaceutically-acceptable salt thereof.

In another aspect, the invention provides a bowel preparation method comprising administering an effective amount of compound 1 and a sufficient quantity of a clear liquid. In yet another aspect, a bowel preparation method of the present invention comprises administering an effective amount of compound 1 and an effective amount of one or more laxative agents selected from an osmotic agent and a stimulant laxative.

The 5-HT₄ receptor agonist, 1-isopropyl-2-oxo-1,2-dihydroquinoline-3-carboxylic acid {(1S,3R,5R)-8-[(R)-2-hydroxy-3-(methanesulfonyl-methyl-amino)propyl]-8-azabicyclo[3.2.1]oct-3-yl}amide having the chemical structure

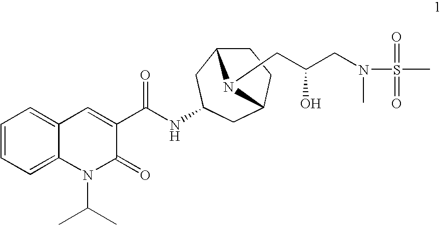

is disclosed in US 2005/0228014.

The present inventors have discovered compound 1 has a rapid and effective prokinetic effect when administered to healthy subjects. Accordingly, the present compound is expected to provide a method of bowel preparation that is more tolerable to patients than the presently available procedures. When a liquid regimen is used in conjunction with the present compound, the liquid regimen is expected to be less demanding than traditional liquid regimens. Alternatively, the present compound is expected be an effective adjunct to bowel preparation regimens by providing prompt bowel evacuation. Use of the present compound is expected to offer more rapid and complete evacuation with greater ease of administration than current regimens, thus promoting better patient compliance.

### DETAILED DESCRIPTION OF THE INVENTION

### Definitions

When describing the methods, compounds, and compositions of the invention, the following terms have the following meanings, unless otherwise indicated.

The term “effective amount” means an amount sufficient to produce the stated effect when administered to a patient.

The term “bowel” is a synonym for intestine and encompasses the small intestine and the large intestine, the latter of which comprises the cecum, the ascending, transverse, and descending colon, and the rectum. The term “bowel cleansing” or equivalently “cleansing of the bowel” or “bowel prep”, as used herein, encompasses “bowel preparation”, “bowel evacuation”, “colon cleansing”, “colonoscopy cleansing”, “colon purgation”, and similar terms, as understood by those skilled in the art.

The term “osmotic agent”, as used herein, includes polyethylene glycol (PEG) electrolyte solutions which are mixtures of sodium sulfate, sodium bicarbonate, sodium chloride, and potassium chloride in an isotonic solution. As a result of the osmotic effect of the polymer, the electrolyte solution is retained in the colon, where it acts as a bowel cleanser. PEG-electrolyte solutions are sold commercially in the United States, for example, as Colyte® and GoLYTELY®, in sulfate-free formulations as NuLYTELY® and TriLyte®, and in low-volume formulations as Halflytely® and Miralax®. Flavoring agents may be included to improve palatability.

The term “osmotic agent” also includes osmotic laxatives, such as sodium phosphate and magnesium citrate. These agents exert their purgative action by osmotically drawing fluid into the intestinal lumen. Sodium phosphate is provided as an aqueous solution, for example, as Fleet® Phospho Soda, and in tablet form as Visicol®. In addition, agents such as glycerin, sorbitol, and mannitol are included in the term osmotic agent.

The term “stimulant laxative” includes agents that produce a laxative effect principally by enhancing bowel wall smooth muscle activity. In addition, they may also increase bowel water content. Stimulant laxatives include bisacodyl, a poorly absorbed diphenylmethane, available commercially, for example under the tradenames Dulcolax®, Fleet® Bisacodyl, and Dulcogen, among others. Stimulant laxatives also include senna laxatives which contain anthraquinone derivatives (glycosides and sennosides) that are activated by colonic bacteria, available, for example, as X-Prep® and Senakot, and sodium picosulfate, provided most commonly in combination with magnesium citrate as Picolax®, as well as phenolphthalein and castor oil.

The term “pharmaceutically-acceptable salt” means a salt prepared from an acid or base which is acceptable for administration to a patient. Such salts can be derived from pharmaceutically-acceptable inorganic or organic acids and from pharmaceutically-acceptable bases. Typically, pharmaceutically-acceptable salts of compounds of the present invention are prepared from acids.

Salts derived from pharmaceutically-acceptable acids include, but are not limited to, acetic, adipic, benzenesulfonic, benzoic, camphorsulfonic, citric, ethanesulfonic, fumaric, gluconic, glutamic, glycolic, hydrobromic, hydrochloric, lactic, maleic, malic, mandelic, methanesulfonic, mucic, nitric, oxalic, pantothenic, phosphoric, succinic, sulfuric, tartaric, p-toluenesulfonic, xinafoic (1-hydroxy-2-naphthoic acid), naphthalene-1,5-disulfonic acid and the like.

It must be noted that, as used in the specification and appended claims, the singular forms “a”, “an”, “one”, and “the” may include plural references, unless the content clearly dictates otherwise.

### Chemical Nomenclature

The prokinetic agent used in the present invention (Compound 1) is designated 1-isopropyl-2-oxo-1,2-dihydroquinoline-3-carboxylic acid {(1S,3R,5R)-8-[(R)-2-hydroxy-3-(methanesulfonyl-methyl-amino)propyl]-8-azabicyclo[3.2.1]oct-3-yl}amide using the commercially-available AutoNom software (MDL Information Systems, GmbH, Frankfurt, Germany). The designation (1S,3R,5R) describes the relative orientation of the bonds associated with the bicyclic ring system. The compound is alternatively denoted as N-[(3-endo)-8-[(R)-2-hydroxy-3-(methanesulfonyl-methyl-amino)propyl]-8-azabicyclo[3.2.1]oct-3-yl]-1-(1-methylethyl)-2-oxo-1,2-dihydro-3-quinolinecarboxamide.

### Synthetic Procedures

Compound 1 can be prepared from readily available starting materials using the procedures described in the Examples below, or using the procedures described in commonly-assigned US 2005/0228014 and US 2006/0183901.

### Pharmaceutical Compositions

The prokinetic agent of the invention is typically administered to a patient in the form of a pharmaceutical composition. Such pharmaceutical compositions may be administered by any acceptable route of administration including, but not limited to, oral, rectal, vaginal, nasal, inhaled, topical (including transdermal) and parenteral modes of administration. Typically, such pharmaceutical compositions will contain from about 0.1 to about 95% by weight of the prokinetic agent; including from about 1 to about 70% by weight, such as from about 5 to about 60% by weight of the prokinetic agent.

Any conventional carrier or excipient may be used in the pharmaceutical compositions comprising the prokinetic agent. The choice of a particular carrier or excipient, or combinations of carriers or excipients, will depend on the intended mode of administration. The preparation of a suitable pharmaceutical composition for a particular mode of administration is well within the scope of those skilled in the pharmaceutical arts. Conventional formulation techniques are described in Remington: The Science and Practice of Pharmacy, 20^{th} Edition, Lippincott Williams & White, Baltimore, Md. (2000); and H. C. Ansel et al., Pharmaceutical Dosage Forms and Drug Delivery Systems, 7^{th} Edition, Lippincott Williams & White, Baltimore, Md. (1999).

Representative examples of materials which can serve as pharmaceutically acceptable carriers include, but are not limited to sugars, starches, cellulose, powdered tragacanth; malt; gelatin; talc; cocoa butter and suppository waxes; oils, glycols, polyols, agar; buffering agents, alginic acid; pyrogen-free water; isotonic saline; Ringer's solution; ethyl alcohol; phosphate buffer solutions; and other non-toxic compatible substances employed in pharmaceutical compositions.

The pharmaceutical compositions comprising the present prokinetic agent are preferably packaged in a unit dosage form. The term “unit dosage form” refers to a physically discrete unit suitable for dosing a patient, i.e., each unit containing a predetermined quantity of active agent calculated to produce the desired therapeutic effect either alone or in combination with one or more additional units. For example, such unit dosage forms may be capsules, tablets, pills, and the like.

Preferably, the present prokinetic agent is provided in a pharmaceutical composition suitable for oral administration. Such pharmaceutical compositions may be in the form of capsules, tablets, pills, lozenges, cachets, dragees, powders, granules; or as a solution or a suspension in an aqueous or non-aqueous liquid; or as an oil-in-water or water-in-oil liquid emulsion; or as an elixir or syrup; and the like; each containing a predetermined amount of a compound of the present invention as an active ingredient.

When intended for oral administration in a solid dosage form (i.e., as capsules, tablets, pills and the like), the pharmaceutical compositions will typically comprise the prokinetic agent and one or more pharmaceutically-acceptable carriers, such as sodium citrate or dicalcium phosphate. Optionally or alternatively, such solid dosage forms may also comprise fillers or extenders, binders, humectants, disintegrating agents, solution retarding agents, absorption accelerators, wetting agents, absorbents, lubricants, coloring agents; and) buffering agents. Release agents, wetting agents, coating agents, sweetening, flavoring and perfuming agents, preservatives and antioxidants can also be present.

The prokinetic agent may be provided in the form of a capsule or cartridge (made, for example, from gelatin) comprising compound 1 or a pharmaceutically-acceptable salt thereof in powdered form and optionally a powdered excipient such as lactose or starch. Additionally, the present agent may be provided in a liquid dosage form, for example, as a pharmaceutically-acceptable emulsion, microemulsion, solution, suspension, syrup or elixir.

### Methods and Preparations of the Invention

The present invention provides a method of cleansing the bowel of a patient in preparation for a diagnostic, surgical, or therapeutic procedure of the colon, the method comprising administering to the patient an effective amount of the prokinetic agent 1-isopropyl-2-oxo-1,2-dihydroquinoline-3-carboxylic acid {(1S,3R,5R)-8-[(R)-2-hydroxy-3-(methanesulfonyl-methyl-amino)propyl]-8-azabicyclo[3.2.1]oct-3-yl}amide or a pharmaceutically-acceptable salt thereof.

In another aspect, the invention provides a method of cleansing the bowel of a patient, the method comprising administering an effective amount of the prokinetic agent 1-isopropyl-2-oxo-1,2-dihydroquinoline-3-carboxylic acid {(1S,3R,5R)-8-[(R)-2-hydroxy-3-(methanesulfonyl-methyl-amino)propyl]-8-azabicyclo[3.2.1]oct-3-yl}amide and a sufficient quantity of a clear liquid.

The invention further provides a method of cleansing the bowel of a patient, the method comprising administering an effective amount of the prokinetic agent 1-isopropyl-2-oxo-1,2-dihydroquinoline-3-carboxylic acid {(1S,3R,5R)-8-[(R)-2-hydroxy-3-(methanesulfonyl-methyl-amino)propyl]-8-azabicyclo[3.2.1]oct-3-yl}amide and an effective amount of one or more laxative agents selected from an osmotic agent and a stimulant laxative.

The dosage of the prokinetic agent used in the present methods will typically be determined by a physician, in the light of the relevant circumstances, including the properties of the specific agents or regimen, if any, used in combination with the present prokinetic agent, and the age, weight, and response of the individual patient.

Suitable doses of the prokinetic agent for bowel preparation are expected to range from about 0.14 to about 1.4 mg/kg of body weight. For an average 70 kg human, this would amount to from about 10 to about 100 mg, including between about 20 and about 70 mg, and between about 40 and about 60 mg of the present prokinetic agent.

Suitable clear liquids for use in the present methods include, but are not limited to, water, chicken broth, fruit juice such as apple juice, white cranberry, or white grape juice, lemonade, fruit flavored beverages, such as Gatorade, and coffee or tea (without cream). A sufficient quantity of a clear liquid is expect to be between about 0.5 liter and about 2.5 liters, including between about 1 and about 2 liters. For example, the evening before the procedure, a patient is directed to abstain from solid food and to consume up to about 0.5 liter of clear liquid other than water and about one liter of water.

In a particular aspect of the invention, the method comprises administering to the patient between about 10 and about 100 mg of 1-isopropyl-2-oxo-1,2-dihydroquinoline-3-carboxylic acid {(1S,3R,5R)-8-[(R)-2-hydroxy-3-(methanesulfonyl-methylamino)propyl]-8-azabicyclo[3.2.1]oct-3-yl}amide or a pharmaceutically-acceptable salt thereof and optionally a suitable quantity of a clear liquid or an effective amount of one or more agents selected from an osmotic agent and a stimulant laxative.

In a particular aspect of the invention, the prokinetic agent is the hydrochloride salt of 1-isopropyl-2-oxo-1,2-dihydroquinoline-3-carboxylic acid {(1S,3R,5R)-8-[(R)-2-hydroxy-3-(methanesulfonyl-methyl-amino)propyl]-8-azabicyclo[3.2.1]oct-3-yl}amide.

In another particular aspect of the invention, the osmotic agent is a PEG electrolyte solution. Typical PEG electrolyte solutions contain the following constituents, per liter: about 60 gram polyethylene glycol 3350, about 1.46 grams sodium chloride, about 0.745 grams potassium chloride, about 1.68 grams sodium bicarbonate, about 5.68 grams sodium sulfate. An effective amount of a PEG electrolyte solution is expected to range from about 2 to about 4 liters.

In an alternative aspect of the invention, the osmotic agent is a sodium phosphate solution or sodium phosphate tablet. Whether provided as an aqueous preparation or in tablet form, effective doses of sodium phosphate typically range from about 40 to about 60 grams sodium phosphate.

In a particular aspect, the stimulant laxative is bisacodyl. Typically, an effective dose of bisacodyl is four tablets each 5 milligrams.

Exemplary regimens for bowel preparation using the present prokinetic agent include, but are not limited to, the following, where compound 1 can be provided as the free base, or, alternatively, as a pharmaceutically-acceptable salt:
Regimen 1: Compound 1 (50 mg)
Regimen 2: Compound 1 (70 mg)
Regimen 3: Compound 1 (40 mg); clear liquid (1 liter)
Regimen 4: Compound 1 (20 mg); clear liquid (2 liters)
Regimen 5: Compound 1 (10 mg); sodium phosphate (6 tablets, total 30 grams) bisacodyl (4 tablets, total 20 milligrams)
Regimen 6: Compound 1 (30 mg); sodium phosphate (6 tablets, total 30 grams) bisacodyl (4 tablets, total 20 milligrams)
Regimen 7: Compound 1 (50 mg); sodium phosphate (6 tablets, total 30 grams) bisacodyl (4 tablets, total 20 milligrams)
Regimen 8. Compound 1 (70 mg); sodium phosphate (6 tablets, total 30 grams) bisacodyl (4 tablets, total 20 milligrams)
Regimen 9: Compound 1 (50 mg); sodium phosphate (6 tablets, total 30 grams)
Regimen 10: Compound 1 (70 mg); sodium phosphate (6 tablets, total 30 grams)
Regimen 11: Compound 1 (30 mg); PEG electrolyte solution (2 liters) bisacodyl (4 tablets, total 20 milligrams)
Regimen 12: Compound 1 (50 mg); PEG electrolyte solution (2 liters) bisacodyl (4 tablets, total 20 milligrams)
Regimen 13: Compound 1 (70 mg); bisacodyl (4 tablets, total 20 milligrams)

To facilitate convenient use of the current preparation methods, the invention further provides a therapeutic package suitable for commercial sale comprising a dosage form of the present prokinetic agent, i.e. a tablet or capsule, and a set of instructions for consumption of clear liquids. In addition, the invention provides a package for commercial sale comprising a dosage form of the present prokinetic agent and a dosage form of an osmotic agent. The osmotic agent may be provided in a conventional format, as a powder which is to be dissolved or suspended in a certain amount of water, as a small volume solution, or in tablet form. The therapeutic package may also contain a stimulant laxative, typically provided in a solid oral dosage form. In another alternative, the therapeutic package comprises a dosage form of the present prokinetic agent and a stimulant laxative.

### CHEMICAL EXAMPLES

The following synthetic examples are offered to illustrate the invention, and are not to be construed in any way as limiting the scope of the invention. In the examples below, the following abbreviations have the following meanings unless otherwise indicated. Abbreviations not defined below have their generally accepted meanings.
Boc=tert-butoxycarbonyl
(Boc)₂O=di-tert-butyl dicarbonate
DCM=dichloromethane
DMF=N,N-dimethylformamide
DMSO=dimethyl sulfoxide
EtOAc=ethyl acetate
mCPBA=m-chlorobenzoic acid
MeCN=acetonitrile
MTBE=tert-butyl methyl ether
PyBop=benzotriazol-1-yloxytripyrrolidino-phosphonium hexafluorophosphate
R_{f}=retention factor
RT=room temperature
TFA=trifluoroacetic acid
THF=tetrahydrofuran

Reagents (including secondary amines) and solvents were purchased from commercial suppliers (Aldrich, Fluka, Sigma, etc.), and used without further purification. Reactions were run under nitrogen atmosphere, unless noted otherwise. Progress of reaction mixtures was monitored by thin layer chromatography (TLC), analytical high performance liquid chromatography (anal. HPLC), and mass spectrometry, the details of which are given below and separately in specific examples of reactions. Reaction mixtures were worked up as described specifically in each reaction; commonly they were purified by extraction and other purification methods such as temperature-, and solvent-dependent crystallization, and precipitation. In addition, reaction mixtures were routinely purified by preparative HPLC. Characterization of reaction products was routinely carried out by mass and ¹H-NMR spectrometry. For NMR measurement, samples were dissolved in deuterated solvent (CD₃OD, CDCl₃, or DMSO-d₆), and ¹H-NMR spectra were acquired with a Varian Gemini 2000 instrument (300 MHz) under standard observation conditions. Mass spectrometric identification of compounds was performed by an electrospray ionization method (ESMS) with an Applied Biosystems (Foster City, Calif.) model API 150 EX instrument or an Agilent (Palo Alto, Calif.) model 1100 LC/MSD instrument. Water content is determined by Karl Fischer titration using a Brinkmann (Westbury, N.Y.) Metrohm Karl Fischer Model 813 coulometer.

### Preparation 1

### (1S,3R,5R)-3-amino-8-azabicyclo[3.2.1]octane-8-carboxylic acid tert-butyl ester

### a. Preparation of 8-benzyl-8-azabicyclo[3.2.1]octan-3-one

Concentrated hydrochloric acid (30 mL) was added to a heterogeneous solution of 2,5-dimethoxy tetrahydrofuran (82.2 g, 0.622 mol) in water (170 mL) while stirring. In a separate flask cooled to 0° C. (ice bath), concentrated hydrochloric acid (92 mL) was added slowly to a solution of benzyl amine (100 g, 0.933 mol) in water (350 mL). The 2,5-dimethoxytetrahydrofuran solution was stirred for approximately 20 min, diluted with water (250 mL), and then the benzyl amine solution was added, followed by the addition of a solution of 1,3-acetonedicarboxylic acid (100 g, 0.684 mol) in water (400 mL) and then the addition of sodium hydrogen phosphate (44 g, 0.31 mol) in water (200 mL). The pH was adjusted from pH 1 to pH ˜4.5 using 40% NaOH. The resulting cloudy and pale yellow solution was stirred overnight. The solution was then acidified to pH 3 from pH 7.5 using 50% hydrochloric acid, heated to 85° C. and stirred for 2 hours. The solution was cooled to room temperature, basified to pH 12 using 40% NaOH, and extracted with dichloromethane (3×500 mL). The combined organic layers were washed with brine, dried (MgSO₄), filtered and concentrated under reduced pressure to produce the crude title intermediate as a viscous brown oil.

To a solution of the crude intermediate in methanol (1000 mL) was added di-tert-butyl dicarbonate (74.6 g, 0.342 mol) at 0° C. The solution was allowed to warm to room temperature and stirred overnight. The methanol was removed under reduced pressure and the resulting oil was dissolved in dichloromethane (1000 mL). The intermediate was extracted into 1 M H₃PO₄ (1000 mL) and washed with dichloromethane (3×250 mL) The aqueous layer was basified to pH 12 using aqueous NaOH, and extracted with dichloromethane (3×500 mL). The combined organic layers were dried (MgSO₄), filtered and concentrated under reduced pressure to produce the title intermediate as a viscous, light brown oil. ¹H-NMR (CDCl₃) δ (ppm) 7.5-7.2 (m, 5H, C₆H₅), 3.7 (s, 2H, CH₂Ph), 3.45 (broad s, 2H, CH—NBn), 2.7-2.6 (dd, 2H, CH₂CO), 2.2-2.1 (dd, 2H, CH₂CO), 2.1-2.0 (m, 2H, CH₂CH₂), 1.6 (m, 2H, CH₂CH₂). (m/z): [M+H]⁺ calcd for C₁₄H₁₇NO 216.14; found, 216.0.

### b. Preparation of 3-oxo-8-azabicyclo[3.2.1]octane-8-carboxylic acid tert-butyl ester

To a solution of 8-benzyl-8-azabicyclo[3.2.1]octan-3-one (75 g, 0.348 mol) in EtOAc (300 mL) was added a solution of di-tert-butyl dicarbonate (83.6 g, 0.383 mol, 1.1 eq) in EtOAc (300 mL). The resulting solution and rinse (100 mL EtOAc) was added to a 1 L Parr hydrogenation vessel containing 23 g of palladium hydroxide (20 wt. % Pd, dry basis, on carbon, ˜50% wet with water; e.g. Pearlman's catalyst) under a stream of nitrogen. The reaction vessel was degassed (alternating vacuum and N₂ five times) and pressurized to 60 psi of H₂ gas. The reaction solution was agitated for two days and recharged with H₂ as needed to keep the H₂ pressure at 60 psi until the reaction was complete as monitored by silica thin layer chromatography. The black solution was then filtered through a pad of Celite® and concentrated under reduced pressure to yield the title intermediate quantitatively as a viscous, yellow to orange oil. It was used in the next step without further treatment. ¹H NMR (CDCl₃) [symbol] (ppm) 4.5 (broad, 2H, CH—NBoc), 2.7 (broad, 2H, CH₂CO), 2.4-2.3 (dd, 2H, CH₂CH₂), 2.1 (broad m, 2H, CH₂CO), 1.7-1.6 (dd, 2H, CH₂CH₂), 1.5 (s, 9H, (CH₃)₃COCON)).

### c. Preparation of (1S,3R,5R)-3-amino-8-azabicyclo[3.2.1]octane-8-carboxylic acid tert-butyl ester

To a solution of the product of the previous step (75.4 g, 0.335 mol) in methanol (1 L) was added ammonium formate (422.5 g, 6.7 mol), water (115 mL) and 65 g of palladium on activated carbon (10% on dry basis, ˜50% wet with water; Degussa type E101NE/W) under a stream of N₂ while stirring via mechanical stirrer. After 24 and 48 hours, additional portions of ammonium formate (132 g, 2.1 mol) were added each time. Once reaction progression ceased, as monitored by anal. HPLC, Celite® (>500 g) was added and the resulting thick suspension was filtered and then the collected solid was rinsed with methanol (˜500 mL). The filtrates were combined and concentrated under reduced pressure until all methanol had been removed. The resulting cloudy, biphasic solution was then diluted with 1M phosphoric acid to a final volume of ˜1.5 to 2.0 L at pH 2 and washed with dichloromethane (3×700 mL). The aqueous layer was basified to pH 12 using 40% aq. NaOH, and extracted with dichloromethane (3×700 mL). The combined organic layers were dried over MgSO₄, filtered, and concentrated by rotary evaporation, then high-vacuum leaving 52 g (70%) of the title intermediate, commonly NBoc-endo-3-aminotropane, as a white to pale yellow solid. The isomer ratio of endo to exo amine of the product was >99 based on ¹H-NMR analysis (>96% purity by analytical HPLC). ¹H NMR (CDCl₃) δ (ppm) 4.2-4.0 (broad d, 2H, CHNBoc), 3.25 (t, 1H, CHNH₂), 2.1-2.05 (m, 4H), 1.9 (m, 2H), 1.4 (s, 9H, (CH₃)₃OCON), 1.2-1.1 (broad, 2H). (m/z): [M+H]⁺ calcd for C₁₂H₂₂N₂O₂ 227.18; found, 227.2. Analytical HPLC (isocratic method; 2:98 (A:B) to 90:10 (A:B) over 5 min): retention time=2.14 min.

### Preparation 2

### 1-isopropyl-2-oxo-1,2-dihydroquinoline-3-carboxylic acid

First, acetone (228.2 mL, 3.11 mol) was added to a stirred suspension of 2-aminophenylmethanol (255.2 g, 2.07 mol) and acetic acid (3.56 mL, 62 mmol) in water (2 L) at room temperature. After 4 h, the suspension was cooled to 0° C. and stirred for an additional 2.5 h and then filtered. The solid was collected and washed with water and the wet solid cooled and dried by lyophilisation to yield 2,2-dimethyl-1,4-dihydro-2H-benzo[1,3]oxazine (332.2 g, 98%) as an off-white solid. ¹H NMR (CDCl₃; 300 MHz): 1.48 (s, 6H, C(CH₃)₂), 4.00 (bs, 1H, NH), 4.86 (s, 2H, CH₂), 6.66 (d, 1H, ArH), 6.81 (t, 1H, ArH), 6.96 (d, 1H, ArH), 7.10 (t, 1H, ArH).

A solution of 2,2-dimethyl-1,4-dihydro-2H-benzo[1,3]oxazine (125 g, 0.77 mol) in THF (1 L) was filtered through a scintillation funnel and then added dropwise via an addition funnel, over a period of 2.5 h, to a stirred solution of 1.0 M LiAlH₄ in THF (800 mL) at 0° C. The reaction was quenched by slow portionwise addition of Na₂SO₄.10H₂O (110 g), over a period of 1.5 h, at 0° C. The reaction mixture was stirred overnight, filtered and the solid salts were washed thoroughly with THF. The filtrate was concentrated under reduced pressure to yield 2-isopropylaminophenylmethanol (120 g, 95%) as a yellow oil. ¹H NMR (CDCl₃; 300 MHz): 1.24 (d, 6H, CH(CH₃)₂), 3.15 (bs, 1H, OH), 3.61 (sept, 1H, CH(CH₃)₂), 4.57 (s, 2H, CH₂), 6.59 (t, 1H, ArH), 6.65 (d, 1H, ArH), 6.99 (d, 1H, ArH), 7.15 (t, 1H, ArH).

Manganese dioxide (85% 182.6 g, 1.79 mol) was added to a stirred solution of 2-isopropylaminophenylmethanol (118 g, 0.71 mol) in toluene (800 mL) and the reaction mixture was heated to 117° C. for 4 h. The reaction mixture was allowed to cool to room temperature overnight and then filtered through a pad of Celite which was eluted with toluene. The filtrate was concentrated under reduced pressure to yield 2-isopropylaminobenzaldehyde (105 g, 90%) as an orange oil. ¹H NMR (CDCl₃; 300 MHz): 1.28 (d, 6H, CH(CH₃)₂), 3.76 (sept, 1H, CH(CH₃)₂), 6.65 (t, 1H, ArH), 6.69 (d, 1H, ArH), 7.37 (d, 1H, ArH), 7.44 (t, 1H, ArH), 9.79 (s, 1H, CHO).

2,2-Dimethyl-[1,3]dioxane-4,6-dione, commonly Meldrum's acid, (166.9 g, 1.16 mol) was added to a stirred solution of 2-isopropylaminobenzaldehyde (105 g, 0.64 mol), acetic acid (73.6 mL, 1.29 mol) and ethylenediamine (43.0 mL, 0.64 mol) in methanol (1 L) at 0° C. The reaction mixture was stirred for 1 h at 0° C. and then at room temperature overnight. The resulting suspension was filtered and the solid washed with methanol and collected to yield the title intermediate, 1-isopropyl-2-oxo-1,2-dihydroquinoline-3-carboxylic acid (146 g, 98%) as an off-white solid. ¹H NMR (CDCl₃; 300 MHz): 1.72 (d, 6H, CH(CH₃)₂), 5.50 (bs, 1H, CH(CH₃)₂), 7.44 (t, 1H, ArH), 7.75-7.77 (m, 2H, ArH), 7.82 (d, 1H, ArH), 8.89 (s, 1H, CH).

### Example 1

### Synthesis of 1-isopropyl-2-oxo-1,2-dihydroquinoline-3-carboxylic acid {(1S,3R,5R)-8-[(R)-2-hydroxy-3-(methanesulfonyl-methylamino)propyl]-8-aza-bicyclo[3.2.1]oct-3-yl}amide

### a. Preparation of (1S,3R,5R)-3-[1-isopropyl-2-oxo-1,2-dihydroquinoline-3-carbonyl)amino]-8-azabicyclo[3.2.1]octane-8-carboxylic acid tert-butyl ester

In a 3 L flask, 1-isopropyl-2-oxo-1,2-dihydroquinoline-3-carboxylic acid (112.4 g, 0.486 mol, 1.1 eq) was suspended in toluene (1 L). The mixture was heated to 85° C. and thionyl chloride (86.74 g, 0.729 mol) was added dropwise over 70 min. The mixture was heated at 95° C. for 1.5 h with stirring and then allowed to cool to room temperature.

In a separate 12 L flask, (1S,3R,5R)-3-amino-8-azabicyclo[3.2.1]octane-8-carboxylic acid tert-butyl ester (100.0 g, 0.442 mol, 1 eq) was suspended in toluene (1 L) and 3 M NaOH (4 eq) was added. The mixture was stirred at room temperature for 10 min and then cooled to about 5° C. The acid chloride solution was added slowly with stirring over 40 min keeping the internal temperature below 10° C. The mixture was stirred at 3-5° C. for 30 min and the layers were allowed to separate overnight. The toluene layer (˜2.5 L) was collected, concentrated to about half (˜1.2 L) by rotary evaporation, and used directly in the next step.

### b. Preparation of 1-isopropyl-2-oxo-1,2-dihydroquinoline-3-carboxylic acid {(1S,3R,5R)-8-azabicyclo[3.2.1]oct-3-yl}amide

To the toluene solution prepared in the previous step (˜1.2 L) was added trifluoroacetic acid (200 mL) over 20 min at 20° C. with stirring. The mixture was stirred at 20° C. for 2 h. Water (1.55 L) was added and the mixture was stirred for 30 min at 20° C. After 30 min, the mixture separated into three layers. The bottom layer (˜350 mL), a viscous brown oil, contained the crude intermediate.

To a 12 L flask charged with MTBE (2.8 L), the crude brown oil was added over 1 h at 1-2° C. with stirring. The suspension was stirred at the same temperature for 1 h and then filtered. The filtrate was washed with MTBE (2×300 mL) and dried under vacuum at room temperature for 4 days to provide the trifluoroacetate salt of the title intermediate (163.3 g) as a pale yellow powder.

### c. Preparation of N-methyl-N—[(S)-2-oxiran-2-ylmethyl]methanesulfonamide

A 12 L flask was charged with water (1 L) followed by the addition NaOH (50% in water, 146.81 g, 1.835 mol). The beaker containing NaOH was washed with water (2×500 mL) and the washings were added to the flask. The mixture was stirred at room temperature for 10 min and cooled to ˜8° C. (N-methyl)methanesulfonamide (200.2 g, 1.835 mol) in water (500 mL) was added over 5 min. The mixture was stirred for 1 h at ˜4° C. and (S)-2-chloromethyloxirane (339.6 g, 3.67 mol) was added. The mixture was stirred for 20 h at 3-4° C. Dichloromethane (2 L) was added and the mixture was stirred for 30 min at 5-10° C. The two layers were allowed to separate over 10 min and collected. The organic layer (˜2.5 L) was added back to the 12 L flask and washed with 1 M H₃PO₄ (800 mL) and brine (800 mL). Dichloromethane was removed by rotary evaporation. To the crude product, toluene (400 mL) was added and removed by rotary evaporation. After three additional cycles of the toluene process, the title intermediate was obtained (228.2 g) which was used without further purification in the next step.

### d. Synthesis of 1-isopropyl-2-oxo-1,2-dihydroquinoline-3-carboxylic acid {(1S,3R,5R)-8-[(R)-2-hydroxy-3-(methanesulfonyl-methyl-amino)propyl]-8-aza-bicyclo[3.2.1]oct-3-yl}amide

In a 3 L flask, 1-isopropyl-2-oxo-1,2-dihydroquinoline-3-carboxylic acid {(1S,3R,5R)-8-azabicyclo[3.2.1]oct-3-yl}amide trifluoroacetate (105.0 g, 0.232 mol) was suspended in absolute ethanol (400 mL). To this suspension, NaOH (50% in water, 0.243 mol. 1.05 eq) dissolved in absolute ethanol (100 mL) was added at room temperature. The beaker containing the NaOH was washed with ethanol (2×50 mL) and the washings were added to the reaction mixture. After 30 min of stirring, a solution of N-methyl-N—[(S)-2-oxiran-2-ylmethyl]methanesulfonamide (62.0 g, 1.5 eq) in absolute ethanol (100 mL) was added. The mixture was refluxed for 2 h, cooled to room temperature and seed crystals of the title compound were added. After about 5 min of stirring a white solid formed. The mixture was cooled to 3-5° C. and stirred for 2 h. The white solid was filtered and the wet cake was washed with cold absolute ethanol (3×50 mL). The solid was dried under vacuum at 30° C. for 60 h to provide the title compound (93.8 g, water content by Karl Fischer method 2.03%). ¹H NMR (CDCl₃) δ ppm 10.52 (d, 1H), 8.83 (s, 1H), 7.75 (d, 2H), 7.64-7.60 (m, 2H), 7.28-7.26 m, 1H), 4.33-4.26 (m, 2H), 3.78-3.75 (m, 1H), 3.27-3.20 (m, 4H), 3.01 (s, 3H), 2.88 (s, 3H), 2.58-2.53 (m, 1H), 2.30-1.81 (m, 1H), 1.68 (d, 6H).

The seed crystals were obtained from a previous preparation of the title compound by the method of this example at smaller scale, in which crystallization occurred spontaneously.

### Example 2

### Synthesis of crystalline hydrochloride salt of 1-isopropyl-2-oxo-1,2-dihydroquinoline-3-carboxylic acid {(1S,3R,5R)-8-[(R)-2-hydroxy-3-(methanesulfonyl-methyl-amino)propyl]-8-aza-bicyclo[3.2.1]oct-3-yl}amide

In a 1 L flask, 1-isopropyl-2-oxo-1,2-dihydroquinoline-3-carboxylic acid {(1S,3R,5R)-8-[(R)-2-hydroxy-3-(methanesulfonyl-methyl-amino)propyl]-8-aza-bicyclo[3.2.1]oct-3-yl}amide (34.7 g, 0.069 mol) was suspended in absolute ethanol (210 mL). Concentrated HCl (1.1 eq) was added at room temperature with stirring. The mixture was stirred at reflux for 30 min and cooled to room temperature and stirred for 2 h. The solid was filtered and the wet cake was washed with cold absolute ethanol (3×50 mL). The solid was dried under vacuum at 30° C. for 48 h to provide the title compound (34.5 g, 93.7% yield, water content by Karl Fischer method 0.13%).

### Clinical Example

### Measurement of Onset of Bowel Movement in Healthy Subjects

Gelatin capsules were filled with up to 10 milligrams (free base equivalent) of 1-isopropyl-2-oxo-1,2-dihydroquinoline-3-carboxylic acid {(1S,3R,5R)-8-[(R)-2-hydroxy-3-(methanesulfonyl-methyl-amino)propyl]-8-aza-bicyclo[3.2.1]oct-3-yl}amide hydrochloride, the present prokinetic agent, prepared according to the method of Examples 1 and 2.

Subjects were dosed weekly with rising doses of the prokinetic agent (0.1 to 70 mg) or placebo in two alternating panels of 10 subjects and a third panel of 10 subjects. Subjects were monitored for safety and evidence of prokinetic effects using a stool diary card. Time and consistency (Bristol score) of each bowel movement were recorded. The median time to first bowel movement and mean number of bowel movements in the first 24 hour post dosing period exhibited a dose dependent effect as shown in Table 1:

TABLE 1

| | Median Time to First | Mean Number of |
|---|---|---|
| Dose | Bowel Movement (hours) | Bowel Movements |
| Placebo | 7.9 | 1.3 (1.4) |
| 10 mg | 1.6 | 2.1 (1.2) |
| 20 mg | 1.0 | 2.0 (1.5) |
| 30 mg | 1.1 | 3.6 (2.4) |
| 50 mg | 1.1 | 5.9 (3.9) |
| 70 mg | 0.9 | 6.0 (3.0) |

*standard deviation in parentheses

On placebo, the average Bristol score (a measure of stool consistency starting at 1 [hard pellets] with a maximum score of 7 [liquid]) was 3.2 (0.7) with a dose dependent increase to a plateau of 5.4-6.0 at 20-70 mg, e.g. at 50 mg the average score was 5.6 (0.9), indicating softer bowel movements. The effect dissipated 24 to 48 hours post dosing.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto. Additionally, all publications, patents, and patent documents cited hereinabove are incorporated by reference herein in full, as though individually incorporated by reference.

What is claimed is:

1. A method of cleansing the bowel of a patient in preparation for a diagnostic, surgical, or therapeutic procedure of the colon, the method comprising administering an effective amount of the prokinetic agent 1-isopropyl-2-oxo-1,2-dihydroquinoline-3-carboxylic acid {(1S,3R,5R)-8-[(R)-2-hydroxy-3-(methanesulfonyl-methyl-amino)propyl]-8-azabicyclo[3.2.1]oct-3-yl}amide or a pharmaceutically-acceptable salt thereof.

2. The method of claim 1 wherein the method further comprises administering a sufficient quantity of a clear liquid.

3. The method of claim 1 wherein the method further comprises administering an effective amount of one or more laxative agents selected from an osmotic agent and a stimulant laxative.

4. The method of claim 3 wherein the prokinetic agent is 1-isopropyl-2-oxo-1,2-dihydroquinoline-3-carboxylic acid {(1S,3R,5R)-8-[(R)-2-hydroxy-3-(methanesulfonyl-methyl-amino)propyl]-8-azabicyclo[3.2.1]oct-3-yl}amide hydrochloride.

5. The method of claim 3 wherein the osmotic agent is a polyethylene glycol electrolyte agent.

6. The method of claim 3 wherein the osmotic agent comprises sodium phosphate.

7. The method of claim 3 wherein the stimulant laxative comprises bisacodyl.

8. The method of claim 3 wherein the one or more laxative agents is an osmotic agent.

9. The method of claim 8 wherein the osmotic agent comprises sodium phosphate.

10. The method of claim 9 wherein the prokinetic agent is 1-isopropyl-2-oxo-1,2-dihydroquinoline-3-carboxylic acid {(1S,3R,5R)-8-[(R)-2-hydroxy-3-(methanesulfonyl-methyl-amino)propyl]-8-azabicyclo[3.2.1]oct-3-yl}amide hydrochloride.

11. The method of claim 3 wherein the one or more laxative agents is a stimulant laxative.

12. The method of claim 11 wherein the stimulant laxative comprises bisacodyl.

13. The method of claim 12 wherein the prokinetic agent is 1-isopropyl-2-oxo-1,2-dihydroquinoline-3-carboxylic acid {(1S,3R,5R)-8-[(R)-2-hydroxy-3-(methanesulfonyl-methyl-amino)propyl]-8-azabicyclo[3.2.1]oct-3-yl}amide hydrochloride.

14. The method of claim 3 wherein the one or more laxative agents is an osmotic agent and a stimulant laxative.

15. The method of claim 14 wherein the osmotic agent comprises sodium phosphate and the stimulant laxative comprises bisacodyl.

16. The method of claim 15 wherein the prokinetic agent is 1-isopropyl-2-oxo-1,2-dihydroquinoline-3-carboxylic acid {(1S,3R,5R)-8-[(R)-2-hydroxy-3-(methanesulfonyl-methyl-amino)propyl]-8-azabicyclo[3.2.1]oct-3-yl}amide hydrochloride.

17. The method of claim 1 wherein the diagnostic procedure is a colonoscopy.

18. The method of claim 10 wherein the diagnostic procedure is a colonoscopy.

19. The method of claim 14 wherein the diagnostic procedure is a colonoscopy.

20. The method of claim 16 wherein the diagnostic procedure is a colonoscopy.
